(19) United States
(12) Patent Application Publication
(10) Pub. No.: US 2017/0029866 A1
(43) Pub. Date: Feb. 2, 2017

(54) METHODS OF MONITORING ADHERENCE TO HALOPERIDOL THERAPY

(71) Applicant: Ameritox, Ltd, Baltimore, MD (US)

(72) Inventors: Erin Strickland, Greensboro, NC (US); Oneka Cummings, Greensboro, NC (US); Gregory L. McIntire, Greensboro, NC (US)

(21) Appl. No.: 15/223,579

(22) Filed: Jul. 29, 2016

Related U.S. Application Data
(60) Provisional application No. 62/198,238, filed on Jul. 29, 2015.

(51) Int. Cl. C12Q 1/40

(52) U.S. Cl. CPC C12Q 1/40; C12Y 302/01031; A61K 31/451

(57) ABSTRACT

The present disclosure provides methods for monitoring subject (e.g., patient) adherence to Haldol therapy, for example as a component of treating a subject for a mental health disorder such as schizophrenia.

### PRIORITY CLAIM

This application claims priority to U.S. Provisional Patent Application Ser. No. 62/198,238, filed Jul. 29, 2015, which is incorporated herein by reference in its entirety.

### TECHNICAL FIELD

The present disclosure provides methods for monitoring subject (e.g., patient) adherence to Haldol (haloperidol and/or haloperidol decanoate) therapy, for example as a component of treating a subject for a mental health disorder such as schizophrenia.

### BACKGROUND

Haloperidol (Haldol®) is a typical antipsychotic prescribed for the treatment of acute symptoms of schizophrenia and a number of additional mental health symptoms including Tourette syndrome and delerium. Drug adherence has been shown to be particularly low in patients with schizophrenia, although Haldol is available in long acting injectable formulas (i.e., Haldol injection and Haldol decanoate injection) which may, in certain circumstances, increase patient adherence. Urine drug testing has been employed by behavioral health clinicians to monitor patient compliance through analysis of drugs and their major metabolites. Typically, adherence to haloperidol therapy is monitored by evaluating levels of haloperidol and one or more metabolites found to be present in urine at approximately 2 and 4% of total dose. In fact, unchanged Haldol has been reported to be present in urine at less than 1% and there is “no evidence of glucuronidation” of the parent drug (Baselt, Disposition of Toxic Drugs and Chemicals in Man, 10th ed. (2014)). These low levels of parent drug and/or metabolites after dosing increase the possibility of false negative monitoring results. Such false negative reports can improperly induce a clinician (e.g., a physician or psychiatrist) to alter a compliant subject's Haldol therapeutic regimen when no alteration is warranted. Improved methods for assessing and monitoring a subject's adherence to Haldol therapy and treating a subject (e.g., a non-adherent subject) on Haldol therapy, are needed.

### SUMMARY

The present disclosure provides methods for monitoring patient adherence to Haloperidol therapy, for example as a component of treating a subject for a mental health disorder such as schizophrenia.

The present disclosure also provides methods of treating a subject on Haldol therapy (e.g., a subject having schizophrenia), the method comprising determining a level of Haldol in fluid associated with the subject after hydrolyzing Haldol glucuronide and/or haloperidol decanoate glucuronide in the fluid with a betaglucuronidase enzyme either naturally occurring (i.e., Abalone derived) or from recombinant sources such as e. coli, and recommending a change in the subject's Haldol therapy if the level of Haldol in the fluid is below a threshold level.

In one embodiment, the present disclosure provides a method for monitoring Haldol therapy in a subject comprising identifying a subject who has been prescribed Haldol therapy, obtaining a fluid sample from the subject, analyzing the fluid sample for the presence of Haldol after hydrolyzing the sample with a betaglucuronidase enzyme either naturally occurring (i.e., Abalone derived) or from recombinant sources such as e. coli and identifying the subject as adherent to the prescribed Haldol therapy if the fluid sample contains Haldol above a threshold level but non-adherent if the fluid sample contains no Haldol or an amount of Haldol below a threshold level.

In another embodiment, the present disclosure provides a method of evaluating compliance with Haldol therapy in a subject, the method comprising obtaining a fluid sample (e.g., urine) from the subject, analyzing the fluid sample after hydrolyzing the sample with a betaglucuronidase enzyme for the presence or absence of an analyte, and identifying the subject as compliant if the analyte is present in the fluid sample.

### DETAILED DESCRIPTION

While the present invention is capable of being embodied in various forms, the description below of several embodiments is made with the understanding that the present disclosure is to be considered as an exemplification of the invention, and is not intended to limit the invention to the specific embodiments illustrated. Headings are provided for convenience only and are not to be construed to limit the invention in any manner. Embodiments illustrated under any heading may be combined with embodiments illustrated under any other heading.

The use of numerical values in the various quantitative values specified in this application, unless expressly indicated otherwise, are stated as approximations as though the minimum and maximum values within the stated ranges were both preceded by the word “about.” Also, the disclosure of ranges is intended as a continuous range including every value between the minimum and maximum values recited as well as any ranges that can be formed by such values. Also disclosed herein are any and all ratios (and ranges of any such ratios) that can be formed by dividing a disclosed numeric value into any other disclosed numeric value. Accordingly, the skilled person will appreciate that many such ratios, ranges, and ranges of ratios can be unambiguously derived from the numerical values presented herein and in all instances such ratios, ranges, and ranges of ratios represent various embodiments of the present invention.

Haloperidol (Haldol®) is a typical antipsychotic prescribed for the treatment of acute symptoms of schizophrenia. Haldol has a molecular weight of 375.9 g/mol, and empirical formula of C₂₁H₂₃ClFNO₂, a pKₐ of 8.3, a logP of 3.66, a CAS number of 52-86-8, a mass-to-charge ratio (m/z) of 376.5 when ionized with addition of a proton (ESI MS), and has a formula shown below:

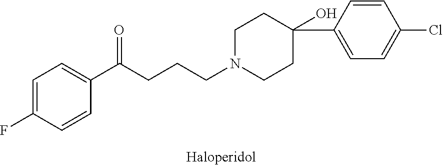 [SMILES: O=C(CCCN1CCC(O)(c2ccc(Cl)cc2)CC1)c1ccc(F)cc1]

Haloperidol is commercially available as tablets (1,2, 5, 10, or 20 mg), an oral solution (2 and 10 mg/mL), an injectable solution (5 mg/mL, max 20 mg/day), and as the injectable haloperidol decanoate (Haldol decanoate) derivative (50 mg/mL), shown below:

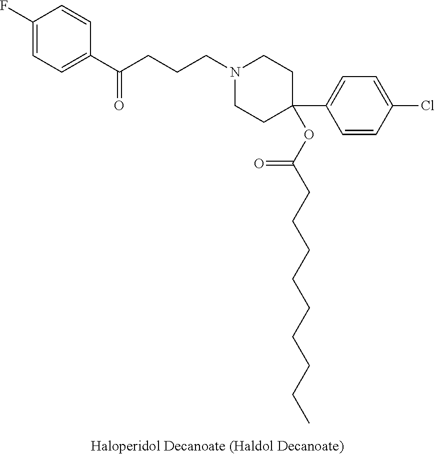 [SMILES: CCCCCCCCCC(=O)OC1(c2ccc(Cl)cc2)CCN(CCCC(=O)c2ccc(F)cc2)CC1]

Drug adherence has been shown to be particularly low in patients with schizophrenia. Urine drug testing has been employed by behavioral health clinicians to monitor patient compliance through analysis of drugs and their major metabolites.

In one embodiment, the present disclosure provides a method for monitoring Haldol therapy in a subject. In some embodiments, the method comprises identifying a subject who has been prescribed Haldol therapy, obtaining a fluid sample from the subject, analyzing the fluid sample after hydrolyzing the sample with a betaglucuronidase enzyme for the presence of Haldol, and identifying the subject as adherent to the prescribed therapy if the fluid sample contains Haldol above a threshold level but non-adherent if the fluid sample contains no Haldol or an amount of Haldol below a threshold level. In some embodiments, the method further comprises counseling the subject on dangers of non-adherence to Haldol therapy if the subject is identified as non-adherent. In some embodiments, the threshold level is a minimum detectable amount of Haldol. In some embodiments, the threshold level is about 5 ng/mL to about 500 ng/mL, for example about 5 ng/mL, about 10 ng/mL, about 15 ng/mL, about 20 ng/mL, about 25 ng/mL, about 30 ng/mL, about 35 ng/mL, about 40 ng/mL, about 45 ng/mL, about 50 ng/mL, about 55 ng/mL, about 60 ng/mL, about 65 ng/mL, about 70 ng/mL, about 75 ng/mL, about 80 ng/mL, about 85 ng/mL, about 90 ng/mL, about 95 ng/mL, about 100 ng/mL, about 125 ng/mL, about 150 ng/mL, about 175 ng/mL, about 200 ng/mL, about 225 ng/mL, about 250 ng/mL, about 275 ng/mL, about 300 ng/mL, about 325 ng/mL, about 350 ng/mL, about 375 ng/mL, about 400 ng/mL, about 425 ng/mL, about 450 ng/mL, about 475 ng/mL, or about 500 ng/mL. In some embodiments, the threshold level is about 50 ng/mL. In some embodiments, the fluid sample is a urine sample.

In another embodiment, the present disclosure provides a method of evaluating compliance with Haldol therapy in a subject. In some embodiments, the method comprises obtaining a fluid sample from the subject, analyzing the fluid sample after hydrolyzing the sample with a betaglucuronidase enzyme for presence or absence of an analyte, and identifying the subject as compliant if the analyte is present in the fluid sample. In some embodiments, the analyte comprises Haldol. In some embodiments, the analyte is considered present in the fluid sample if the analyte is detected above a threshold value. In some embodiments, the threshold value is about 50 ng/mL. In other embodiments, the threshold value is about 25 ng/mL. In still other embodiments, the threshold value is about 5 ng/mL.

In any embodiment described herein, the method may further comprise counseling the subject on dangers of non-adherence or non-compliance to Haldol therapy if the subject is identified as non-adherent or non-compliant.

In any embodiment described herein, the method may further comprise generating a report including a statement indicating whether the subject is identified as adherent or non-adherent, or as compliant or non-compliant, to the haloperidol therapy. In some embodiments, the report further includes a recommendation to alter the haloperidol therapy if the subject is identified as non-adherent or non-compliant.

In some embodiments, the present disclosure provides a method of treating a subject on Haldol therapy, the method comprising determining a level of Haldol in fluid associated with the subject after hydrolyzing Haldol glucuronide and/or haloperidol decanoate glucuronide in the fluid with a betaglucuronidase enzyme either naturally occurring (i.e., Abalone derived) or from recombinant sources such as e. coli, and recommending a change in the subject's Haldol therapy if the level of Haldol in the fluid is below a threshold level. In some embodiments, the subject has schizophrenia. In some embodiments, the Haldol therapy comprises, consists essentially of, or consists of administration of haloperidol. In some embodiments, the Haldol therapy comprises, consists essentially of, or consists of administration of haloperidol decanoate.

In some embodiments, the present disclosure provides a method of treating a subject on Haldol therapy, the method comprising contacting a fluid associated with the subject with a betaglucuronidase enzyme, thereafter determining a level of Haldol in the fluid, and recommending a change in the subject's Haldol therapy if the level of Haldol in the fluid is below a threshold level. In some embodiments, the subject has schizophrenia. In some embodiments, the Haldol comprises, consists essentially of, or consists of administration of haloperidol. In some embodiments, the Haldol comprises, consists essentially of, or consists of administration of haloperidol decanoate.

### EXAMPLES

### Example 1

Urine samples of normally metabolizing human subjects who were known to be taking chronic doses of Haldol were tested for the presence of Haldol before and after hydrolysis with a betaglucuronidase enzyme.

Surprisingly, the amount of Haldol determined in these samples increased post hydrolysis. This is surprising inasmuch as the literature teaches that there is no evidence of Haldol glucuronidation (e.g., Baselt, 10^{th} ed.). Thus, the present disclosure provides methods of analyzing Haldol adherence or compliance comprising analyzing a fluid sample of the subject for Haldol and/or a Haldol metabolite, the method comprising contacting the fluid sample with a hydrolysis agent. The hydrolysis agent may be any suitable hydrolysis agent, such as an acid, a base, or an enzyme to free significant amounts of Haldol parent drug for analysis.

TABLE 1
Data analysis pre and post hydrolysis

| | Pre Hydrolysis | | Post hydrolysis | |
|---|---|---|---|---|
| Sample ID | Method (ng/mL) | Postives | Method (ng/mL) | Positives |
| 1 | 8.0 | Positive | 1473.4 | Positive |
| 2 | 0.0 | | 0.0 | |
| 3 | 0.0 | | 123.1 | Positive |
| 4 | 0.0 | | 773.0 | Positive |
| 5 | 0.0 | | 0.0 | |
| 6 | 0.0 | | 0.0 | |
| 7 | 0.0 | | 0.0 | |
| 8 | 10.0 | Positive | 731.3 | Positive |
| 9 | 0.0 | | 64.4 | Positive |
| 10 | 0.0 | | 0.0 | |
| 11 | 5.0 | Positive | 288.7 | Positive |
| 12 | 0.0 | | 284.0 | Positive |
| 13 | 0.0 | | 599.7 | Positive |
| 14 | 6.0 | Positive | 673.3 | Positive |
| 15 | 13.0 | Positive | 290.1 | Positive |
| 16 | 0.0 | | 354.5 | Positive |
| 17 | 0.0 | | 212.7 | Positive |
| 18 | 8.0 | Positive | 728.3 | Positive |
| 19 | 9.0 | Positive | 180.5 | Positive |
| 20 | 0.0 | | 187.2 | Positive |
| 21 | 0.0 | | 0.0 | |
| 22 | 0.0 | | 0.0 | |
| 23 | 0.0 | | 0.0 | |
| 24 | 0.0 | | 0.0 | |
| 25 | 0.0 | | 0.0 | |
| 26 | 0.0 | | 1037.6 | Positive |
| 27 | 0.0 | | 280.2 | Positive |
| 28 | 0.0 | | 0.0 | |
| 29 | 0.0 | | 78.1 | Positive |
| 30 | 10.0 | Positive | 609.6 | Positive |
| 31 | 0.0 | | 0.0 | |
| 32 | 5.0 | Positive | 43.5 | Positive |
| 33 | 13.0 | Positive | 654.9 | Positive |
| 34 | 0.0 | | 282.2 | Positive |
| 35 | 12.0 | Positive | 675.1 | Positive |
| 36 | 414.0 | Positive | 3998.3 | Positive |
| 37 | 154.0 | Positive | 4499.0 | Positive |
| 38 | 9.0 | Positive | 221.1 | Positive |
| 39 | 0.0 | | 518.8 | Positive |
| 40 | 0.0 | | 507.8 | Positive |
| 41 | 165.0 | Positive | 1224.5 | Positive |
| 42 | 168.0 | Positive | 966.8 | Positive |
| 43 | 156.0 | Positive | 4396.2 | Positive |
| 44 | 0.0 | | 807.9 | Positive |
| 45 | 14.0 | Positive | 939.6 | Positive |
| 46 | 0.0 | | 590.6 | Positive |
| 47 | 5.0 | Positive | 763.2 | Positive |
| 48 | 113.0 | Positive | 2089.5 | Positive |
| 49 | 0.0 | | 0.0 | |
| 50 | 230.0 | Positive | 2988.4 | Positive |
| 51 | 8.0 | Positive | 932.3 | Positive |
| 52 | 52.0 | Positive | 4642.1 | Positive |
| 53 | 4.0 | | 343.0 | Positive |
| 54 | 0.0 | | 253.7 | Positive |
| 55 | 0.0 | | 0.0 | |
| 56 | 0.0 | | 0.0 | |
| 57 | 0.0 | | 871.8 | Positive |
| 58 | 8.0 | | 1063.4 | Positive |
| 59 | 0.0 | | 40.8 | Positive |
| 60 | 0.0 | | 5.0 | Positive |
| 61 | 0.0 | | 136.3 | Positive |
| 62 | 82.0 | Positive | 1636.2 | Positive |
| 63 | 0.0 | | 226.7 | Positive |
| 64 | 24.0 | Positive | 768.0 | Positive |
| 65 | 0.0 | | 32.8 | Positive |
| 66 | 238.0 | Positive | 16805.6 | Positive |
| 67 | 7.0 | Positive | 183.3 | Positive |
| 68 | 2.0 | | 145.5 | Positive |
| 69 | 9.0 | Positive | 527.5 | Positive |
| 70 | 0.0 | | 0.0 | |
| 71 | 6.0 | | 181.9 | Positive |
| 72 | 0.0 | | 0.0 | |
| 73 | 257.0 | Positive | 3144.7 | Positive |
| 74 | 0.0 | | 0.0 | |
| 75 | 67.0 | Positive | 2555.4 | Positive |
| 76 | 68.0 | Positive | 5591.3 | Positive |
| 77 | 0.0 | | 0.0 | |
| 78 | 0.0 | | 135.1 | Positive |
| 79 | 16.0 | Positive | 661.7 | Positive |
| 80 | 22.0 | Positive | 382.3 | Positive |
| 81 | 32.0 | Positive | 1550.5 | Positive |
| 82 | 22.0 | Positive | 2403.1 | Positive |
| 83 | 24.0 | Positive | 218.1 | Positive |
| 84 | 24.0 | Positive | 1813.7 | Positive |
| 85 | 48.0 | Positive | 864.6 | Positive |
| 86 | 69.0 | Positive | 9669.6 | Positive |
| 87 | 41.0 | Positive | 966.5 | Positive |
| 88 | 38.0 | Positive | 282.1 | Positive |
| 89 | 35.0 | Positive | 42.8 | Positive |
| 90 | 5.0 | Positive | 661.2 | Positive |
| 91 | 65.0 | Positive | 1351.0 | Positive |
| 92 | 0.0 | | 33.3 | Positive |
| 93 | 5.0 | Positive | 124.5 | Positive |
| 94 | 88.0 | Positive | 2787.1 | Positive |
| 95 | 0.0 | | 624.8 | Positive |
| 96 | 0.0 | | 929.3 | Positive |
| 97 | 0.0 | | 31.8 | Positive |
| 98 | 0.0 | | 0.0 | |
| 99 | 37.0 | Positive | 186.1 | Positive |
| 100 | 0.0 | | 0.0 | |
| 101 | 0.0 | | 0.0 | |
| 102 | 61.0 | Positive | 2025.2 | Positive |
| 103 | 66.0 | Positive | 3017.1 | Positive |
| 104 | 9.0 | Positive | 143.4 | Positive |
| 105 | 0.0 | | 0.0 | |
| 106 | 0.0 | | 80.2 | Positive |
| 107 | 25.0 | Positive | 1082.4 | Positive |
| 108 | 21.0 | Positive | 515.9 | Positive |
| 109 | 0.0 | | 420.8 | Positive |
| 110 | 0.0 | | 197.7 | Positive |
| 111 | 43.0 | Positive | 770.9 | Positive |
| 112 | 17.0 | Positive | 540.9 | Positive |
| 113 | 0.0 | | 0.0 | |
| 114 | 15.0 | Positive | 176.4 | Positive |
| 115 | 4.0 | | 289.5 | Positive |
| 116 | 0.0 | | 46.7 | Positive |
| 117 | 0.0 | | 39.3 | Positive |
| 118 | 0.0 | | 0.0 | |
| 119 | 12.0 | Positive | 858.2 | Positive |
| 120 | 0.0 | | 0.0 | |
| 121 | 65.0 | Positive | 2084.1 | Positive |
| 122 | 0.0 | | 0.0 | |
| 123 | 19.0 | Positive | 640.0 | Positive |
| 124 | 0.0 | | 0.0 | |
| 125 | 147.0 | Positive | 2752.7 | Positive |
| 126 | 0.0 | | 99.3 | Positive |
| 127 | 24.0 | Positive | 2145.4 | Positive |
| 128 | 0.0 | | 205.4 | Positive |
| 129 | 25.0 | Positive | 2502.5 | Positive |
| 130 | 13.0 | Positive | 326.3 | Positive |
| 131 | 83.0 | Positive | 1488.1 | Positive |
| 132 | 0.0 | | 60.5 | Positive |
| 133 | 68.0 | Positive | 2235.1 | Positive |
| 134 | 10.0 | Positive | 316.0 | Positive |
| 135 | 7.0 | Positive | 128.6 | Positive |
| 136 | 0.0 | | 146.3 | Positive |
| 137 | 0.0 | | 0.0 | |
| 138 | 79.0 | Positive | 2138.0 | Positive |
| 139 | 34.0 | Positive | 1582.0 | Positive |
| 140 | 355.0 | Positive | 4890.9 | Positive |
| 141 | 0.0 | | 147.4 | Positive |
| 142 | 31.0 | Positive | 291.5 | Positive |
| 143 | 115.0 | Positive | 4713.0 | Positive |
| 144 | 0.0 | | 0.0 | |
| 145 | 26.0 | Positive | 937.0 | Positive |
| 146 | 0.0 | | 122.1 | Positive |
| 147 | 9.0 | Positive | 1526.6 | Positive |
| 148 | 313.0 | Positive | 5329.9 | Positive |
| 149 | 13.0 | Positive | 400.5 | Positive |
| 150 | 0.0 | | 0.0 | |
| 151 | 0.0 | | 0.0 | |

As shown above, parent drug Haldol was unexpectedly increased in detectable quantities in many tests of subject's urine samples. This was unexpected because the parent drug (Haldol) is reported to be less than 1% of dose in the urine and to not be glucuronidated (Baselt, 10th ed.). Notably, reliance on the presence of haloperidol without hydrolysis would have generated false negative test results for at least half of the subjects listed in Table 1, as shown in Table 2.

TABLE 2
Summary of data from Table 1.

| Parameter | Without Hydrolysis | With hydrolysis |
|---|---|---|
| N | 151 | 151 |
| Min | 0.0 | 0.0 |
| Max | 414.0 | 16805.6 |
| Avg | 30.7 | 982.5 |
| Std Dev | 66.7 | 1907.3 |
| total positive | 75 | 119 |
| total negative | 76 | 32 |
| % positive | 49.67 | 78.81 |

These data demonstrate that hydrolysis before analysis of haloperidol in urine samples provides a greater level of sensitivity and consistency among subjects on Haldol therapy, and therefore provides a superior urine analyte for evaluation of a subject's compliance with a Haldol therapeutic regimen.

### Example 2.

The data in example 1 were not separated by dose type, however, a small sample number of each dose type reveals the data presented in Table 3, wherein patients using the injectables (i.e., solution and decanoate derivative) are the most “adherent” as expected. Those on tablets are likely less adherent but still above the case where hydrolysis is not utilized. However, those patients taking the oral solution are likely extremely non-adherent. Again, this is from a sample of data for various dosage forms of haloperidol following beta-glucuronidase hydrolysis to determine Haldol® levels in urine.

TABLE 3
Impact of Hydrolysis on % Positive Patients by Dosage Form

| | | | % | Failed |
|---|---|---|---|---|
| Dose type | Negative | Positive | Positive | SVT* |
| Injectable Haloperidol Decanoate | 0/20 | 20/20 | 100% | 2 |
| Injectable Haloperidol Solution | 3/35 | 32/35 | 94% | 3 |
| Haloperidol Tablets | 26/181 | 155/181 | 85% | 8 |
| Haloperidol Oral Solution | 73/106 | 33/106 | 31% | 3 |

*Specimen Validity Testing (pH, specific gravity, and creatinine concentration

Analysis of a different set of samples without hydrolysis (historical data collected using the previous method where hydrolysis was not employed) is shown in Table 4 and suggests that without hydrolysis, a significant number of “false negatives” are observed no matter what the dosage pathway. Obviously, suggesting that only 64% and 66% of patients who have been injected with drug are adherent leaves room to question the analytical method and hence the search for the glucuronide conjugate. The impact of hydrolysis for adequate detection of this drug is irrefutably demonstrated in Table 3 where the injectable haloperidol solution is detected in 94% of patients tested while the haloperidol injectable decanoate which is usually administered because of its extended effect is detected in 100% of the patients tested.

TABLE 4
% Positive Patients by Dosage Form without Hydrolysis

| | | | | Failed |
|---|---|---|---|---|
| Dose type | Negative | Positive | % Positive | SVT* |
| Injectable Haloperidol | 17/56 | 36/56 | 64% | 3 |
| Decanoate | | | | |
| Injectable Haloperidol | 59/178 | 119/178 | 66% | 14 |
| Solution | | | | |
| Haloperidol Tablets | 284/646 | 362/646 | 56% | 45 |
| Haloperidol Oral Solution | N/A | N/A | N/A | N/A |

From the foregoing, it will be appreciated that specific embodiments of the invention have been described herein for purposes of illustration, but that various modifications may be made without deviating from the scope of the invention. Accordingly, the invention is not limited except as by the appended claims.

I/We claim:

1. A method for monitoring haloperidol (Haldol) therapy in a subject comprising:
identifying a subject who has been prescribed Haldol therapy;
obtaining a fluid sample from the subject;
hydrolyzing the sample;
analyzing the fluid sample for the presence of haloperidol; and
identifying the subject as adherent to the prescribed Haldol therapy if the hydrolysed fluid sample contains Haldol above a threshold level but non-adherent if the hydrolysed fluid sample contains no Haldol or an amount of Haldol below a threshold level.

2. The method of claim 1 further comprising counseling the subject on dangers of non-adherence to Haldol therapy if the subject is identified as non-adherent.

3. The method of claim 1 wherein the threshold level is a minimum detectable amount of Haldol.

4. The method of claim 1, wherein the threshold level is about 50 ng/mL, more preferably about 20 ng/mL and most preferably about 5 ng/mL.

5. The method of claim 1, wherein the fluid sample is a urine sample.

6. The method of claim 1 further comprising generating a report including a statement indicating whether the subject is identified as adherent or non-adherent to the haloperidol therapy.

7. The method of claim 6, wherein the report further includes a recommendation to alter the haloperidol therapy if the subject is identified as non-adherent.

8. A method of evaluating compliance with Haldol therapy in a subject, the method comprising:
obtaining a fluid sample from the subject;
hydrolyzing the sample;
analyzing the fluid sample for presence or absence of an analyte; and
identifying the subject as compliant if the analyte is present in the fluid sample.

9. The method of claim 8, wherein the analyte comprises Haldol.

10. The method of claim 9, wherein the analyte results from hydrolysis by betaglucuronidase.

11. The method of claim 9, wherein the analyte comprises Haldol.

12. The method of claim 8, wherein the analyte is considered present in the fluid sample if the analyte is detected above a threshold value.

13. The method of claim 12, wherein the threshold value is about 50 ng/mL, more preferably about 20 ng/mL, and most preferably about 5 ng/mL.

14. The method of claim 8 further comprising generating a report including a statement indicating whether the subject is identified as compliant or non-compliant to the haloperidol therapy.

15. The method of claim 14, wherein the report further includes a recommendation to alter the haloperidol therapy if the subject is identified as non-compliant.
